(19) United States
(12) Patent Application Publication
(10) Pub. No.: US 2022/0211540 A1
(43) Pub. Date: Jul. 7, 2022

(54) PENILE PROPHYLACTIC DEVICE WITH FLAVOR RIDGE FOR GUIDED FELLATIO

(71) Applicants: Theresa Marie Hernandez, Sacramento, CA (US); Melissa Mae Andrews, Reno, NV (US); Jeffrey Dean Parker, Othello, WA (US); Russ Weinzimmer, Milford, NH (US)

(72) Inventors: Theresa Marie Hernandez, Sacramento, CA (US); Melissa Mae Andrews, Reno, NV (US); Jeffrey Dean Parker, Othello, WA (US); Russ Weinzimmer, Milford, NH (US)

(21) Appl. No.: 17/398,759

(22) Filed: Aug. 10, 2021

Related U.S. Application Data
(63) Continuation-in-part of application No. 17/141,878, filed on Jan. 5, 2021.

(51) Int. Cl. A61F 6/04

(52) U.S. Cl. CPC A61F 6/04; A61F 2006/048

(57) ABSTRACT

A penile prophylactic device for enhancing fellatio by providing guided licking as directed by a flavor ridge running along a top portion and/or a bottom portion of the device. The device can be made from a polymeric material, such as LDPE. The flavor ridge serves to provide tactile guidance to the tongue of the person licking the penis covered by the prophylactic device, such that the wearer of the device experiences pleasurable sensations while the flavor ridge is licked along its length. Optionally attached to the opening of the prophylactic device is an elastic band configured to securely hold the opening of the device in place along a proximal portion of the penis. The elastic band can be stretched so as to be constrict around a proximal portion of the penis. The device empowers both giver and receiver of fellatio to enjoy enhanced sensations, while being protected from STD transmission.

### CROSS-REFERENCE TO RELATED APPLICATIONS

The present application is a Continuation-in-Part of U.S. patent application Ser. No. 17/141,878, filed Jan. 5, 2021, titled “SCROTUM PROPHYLACTIC DEVICE WITH A FLAVOR STRIP”, which is hereby incorporated by reference in its entirety.

### FIELD OF THE INVENTION

This invention relates generally to prophylactic devices for protecting a portion of human male genitalia, and specifically to prophylactic devices for providing a barrier against disease transmission to or from a human male penis.

### BACKGROUND OF THE INVENTION

An important reason why many women feel uncomfortable giving oral sex to a man—particularly when with a new sexual partner—is concern about sexually transmitted diseases. Today, many men are also concerned about exposing their partner to disease transmission during intimate contact.

Often when one engages in oral sex, one does not know the disease status of one's partner. Contrary to common belief, disease transmission during oral sex is a very real danger. For example, oral transmission of herpes simplex, syphilis, gonorrhea, chlamydia, and a host of other lesser-known STDs is possible.

### SUMMARY OF THE INVENTION

The invention is a penile prophylactic device for guided fellatio that protects the penis from bodily fluids, such as saliva, and orally transmitted STDs, and protects the giver of fellatio from being exposed to an STD, and/or ejaculate. The prophylactic device includes a closed-end sleeve that is sized and shaped so as to cover most of a typical human male penis, the closed-end sleeve being made from a polymeric material, such as low-density polyethylene (LDPE), and optionally includes an elastic band around the opening. The LDPE can be clear, and of the same thickness as used to make plastic wrap for use with food. The majority of plastic wrap sold to consumers is 8 μm, 9 μm, or 10 μm thick. Thinner plastic wrap transmits body heat better than thicker plastic wrap, but thicker plastic wrap resists tearing, breaking, and or ripping.

The elastic band can be made from an elastomer, such as silicone. When the prophylactic device includes an elastic band, the prophylactic device has an opening that functions like the opening of a shower cap, i.e., that keeps the prophylactic device in place by being stretched open and then constricting around the base of the penis.

The prophylactic device enables the giver and the receiver of fellatio (oral stimulation of the penis) to enjoy the experience, without worrying about STD transmission. For example, the material of the closed-end sleeve allows the male recipient of oral stimulation of the penis to feel the probing and warmth of the giver's tongue, without encountering the STD transmission risk of direct tongue-to-genital contact. Thus, the device of the invention provides reduction in STD transmission without reducing the enjoyment of fellatio.

The flavor ridge of the prophylactic device of the invention enables and encourages the giver of fellatio to safely engage more in oral sex, including licking along the shaft of the penis, and/or sucking the penis.

The prophylactic device of the invention provides a prophylactic/erotic enhancement breakthrough—one that serves the needs and preferences of both women and men.

A flavor strip running along the prophylactic device of the invention allows a woman to perform fellatio while enjoying the aroma and taste of the flavor strip, such as cherry, vanilla, chocolate, raspberry, or other generally pleasant flavor. Meanwhile, her male partner derives most of the pleasures of receiving oral sex, including feeling the warmth of tactile stimulation, without any concerns about disease transmission to or from his penis.

The prophylactic device of the invention is also useful for gay and bisexual men.

The prophylactic device of the invention is primarily for oral use, and is NOT intended to be inserted anally or vaginally.

Further, the prophylactic device of the invention is NOT intended for use as a birth control device, and is not intended to prevent pregnancy.

A general aspect of the invention is a penile prophylactic device for guided fellatio. The penile prophylactic device includes: a polymeric sleeve having an open end and a closed end, the polymeric sleeve configured to substantially cover a penis of a human male; and a flavor ridge running along a surface of the polymeric sleeve.

In some embodiments, the flavor ridge runs along a top surface and/or a bottom surface of the polymeric sleeve.

In some embodiments, the polymeric sleeve is made from a polymeric material selected from one of: low-density polyethylene (LDPE) or polyurethane or polyisoprene.

In some embodiments, the penile prophylactic device further includes: an elastic band attached around the circumference of the opening of the polymeric sleeve, the elastic band configured to constrict around a portion of a penis proximal to the scrotum, thereby helping to hold the polymeric sleeve in place along the penis.

In some embodiments, the elastic band is made of silicone.

In some embodiments, the flavor ridge includes at least one of: a sweetening agent, a souring agent, and a flavoring agent.

In some embodiments, the penile prophylactic device further includes: a protective strip removably attached along the flavor ridge, the protective strip being configured to protect freshness of the flavor ridge while the at least one protective strip is attached to the flavor ridge, and configured to be removable so as to leave the flavor ridge substantially in place after the protective strip is removed.

In some embodiments, the flavor ridge includes a pair of mutually parallel flavor ridges on the polymeric sleeve.

In some embodiments, the penile prophylactic device further includes: a backing layer configured to adhere to the polymeric sleeve, the backing layer applied centrally along the polymeric sleeve, the flavor ridge being applied centrally along the backing layer.

In some embodiments, the backing layer includes a coating of light adhesive that improves the adhesion of the backing layer to the polymeric sleeve.

In some embodiments, the backing layer includes a smooth front-facing surface that is configured to retain its shape while the flavor ridge off is licked off of the backing layer.

In some embodiments, the device is configured to be packaged in a folded condition.

In some embodiments, the flavor ridge is folded once or twice when the device is packaged in the folded condition.

In some embodiments, the flavor ridge is flavored to resemble at least one of: lemon, lime, orange, cherry, pineapple, grape, blueberry, licorice, chocolate, cotton candy.

Another general aspect of the invention is a penile prophylactic device for enhancing fellatio, this device including: a polymeric sleeve having an open end and a closed end, the polymeric sleeve configured to substantially cover a penis of a human male; a flavor ridge running along a top surface and/or a bottom surface of the polymeric sleeve; and an elastic band attached around the circumference of the opening of the polymeric sleeve, the elastic band configured to constrict around a portion of a penis proximal to the scrotum, thereby helping to hold the polymeric sleeve in place along the penis.

In some embodiments, the polymeric sleeve is made from a polymeric material selected from one of: low-density polyethylene (LDPE) or polyurethane or polyisoprene.

In some embodiments, the elastic band is made of silicone. In some embodiments, the flavor ridge includes at least one of: a sweetening agent, a souring agent, and a flavoring agent.

In some embodiments, the penile prophylactic device further includes: a protective strip removably attached along the flavor ridge, the protective strip being configured to protect freshness of the flavor ridge while the at least one protective strip is attached to the flavor ridge, and configured to be removable so as to leave the flavor ridge substantially in place after the protective strip is removed.

### BRIEF DESCRIPTION OF THE DRAWINGS

The invention will be more fully understood from the following Detailed Description, in conjunction with the following figures, wherein:

FIG. 1 is a side view of the prophylactic device worn on a penis of a human male, the device having a top flavor ridge for guided fellatio running centrally along a distal portion of the top surface of the device, and a bottom flavor ridge running centrally along the entire bottom surface of the device, the prophylactic device also having an elastic band secured to the opening of the device.

FIG. 2 is a side view of another embodiment of the prophylactic device of FIG. 1, showing a flavor ridge running centrally along a distal portion of the top surface of the prophylactic device, and continuing to run centrally along a distal portion of the bottom surface of the prophylactic device.

FIG. 3 is a side view of yet another embodiment of a prophylactic device according to the invention, this embodiment not having an elastic band secured to the opening of the prophylactic device, showing a first flavor ridge running centrally along a distal portion of the top surface of the device, and continuing to run centrally along a distal and medial portion of the bottom surface of the device, also including a second flavor ridge running along the left and the right sides of the prophylactic device.

FIG. 4 is a side view of a prophylactic device having a flavor ridge running centrally along a right side of the device, the flavor ridge covered by a protective strip, the prophylactic device also having an elastic band secured to the opening of the device.

FIG. 5 is a side view of the prophylactic device of FIG. 4, showing the protective strip being peeled off of the flavor ridge prior to licking along the flavor ridge.

FIG. 6 is a top view of the device of FIG. 4, the prophylactic device shown laid flat on a surface, and arranged so as to show the flavor ridge running centrally along the entire length of the device, the protective strip removably adhering to and covering the flavor ridge.

### DETAILED DESCRIPTION

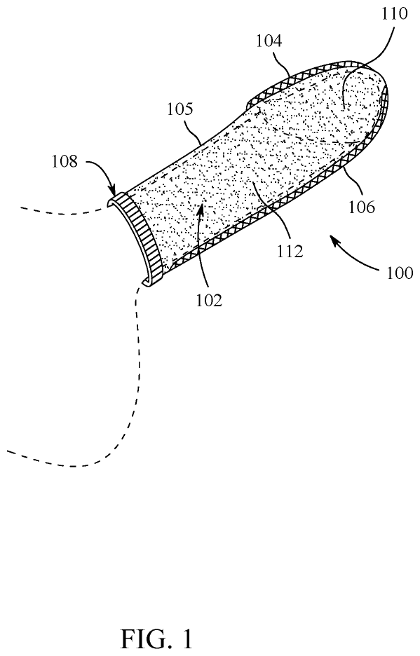

With reference to FIG. 1, a side view is shown of an embodiment of the prophylactic device 100 of the invention worn on a penis 102 of a human male. The prophylactic device 100 has a top flavor ridge 104 for guided fellatio running centrally along a distal portion of the top surface of the polymeric sleeve 105 of the device 100, and a bottom flavor ridge 106 running centrally along the entire bottom surface of the polymeric sleeve 105 of the device 100, the device 100 also having an elastic band 108 secured circumferentially about the opening of the polymeric sleeve 105 of the device 100. The top flavor ridge 104 runs centrally over the top of the glans 110 of the penis 102 for guided fellatio of the glans 110, and the bottom flavor ridge 106 runs centrally along the entire frenum 112 of the penis 102 for guided licking along the frenum 112, which runs along the bottom of the penis 100.

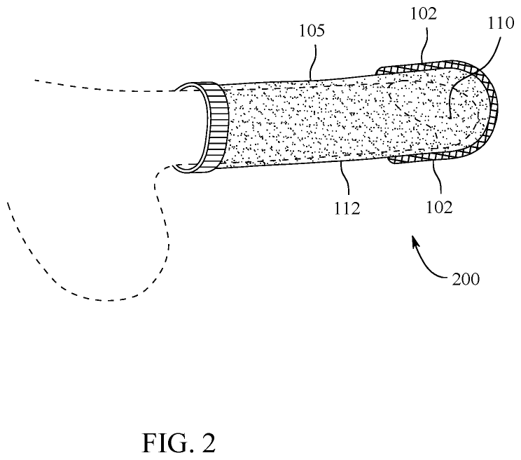

Regarding FIG. 2, a side view is shown of another embodiment 200 of the prophylactic device of the invention, having a flavor ridge 102 running centrally along a distal portion of the top surface of the polymeric sleeve 105 of the device 102, and continuing to run centrally along a distal portion of the bottom surface of the polymeric sleeve 105 of the device 200. This flavor ridge 102 provides guided licking centrally from the top of the glans 110 to the bottom of the glans 110, and on to the distal portion of the frenum 112.

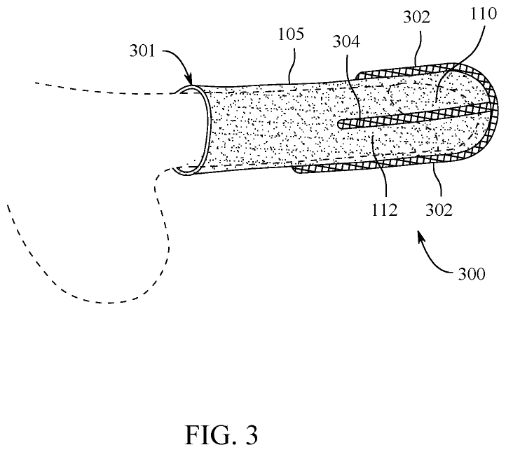

With reference to FIG. 3, a side view is presented of yet another embodiment 300 of the device of the invention, this embodiment 300 not having an elastic band secured to the opening 301 of the polymeric sleeve 105 of the device 300. A first flavor ridge 302 runs centrally along a distal portion of the top surface of the polymeric sleeve 105 of the device 300, and continues to run centrally along a distal and medial portion of the bottom surface of the polymeric sleeve 105 of the device 300, also including a second flavor ridge 304 running along the left and the right sides of the polymeric sleeve 105 of the device 300. These flavor ridges 302 and 304 provide guided licking centrally from the top of the glans 110 to the bottom of the glans 110 and a distal portion of the frenum 112, as well as along the right and left distal portions of the glans 110.

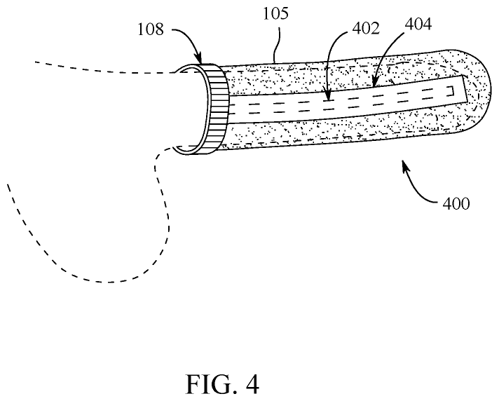

Referring to FIG. 4, a side view is shown of an embodiment 400 of the prophylactic device of the invention having a flavor ridge 402 running centrally along a right side of the polymeric sleeve 105 of the device 400, the flavor ridge 402 covered by a protective strip 404, the device 400 also having an elastic band 108 secured to the opening of the polymeric sleeve 105 of the device 400.

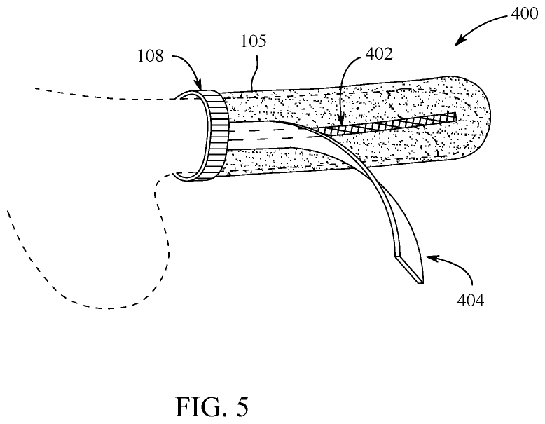

Referring to FIG. 5, a side view is shown of the prophylactic device embodiment 400 of FIG. 4, showing the protective strip 404 being peeled off of the flavor ridge 402 (and possibly also off of the polymeric sleeve 105) prior to licking along the flavor ridge 402.

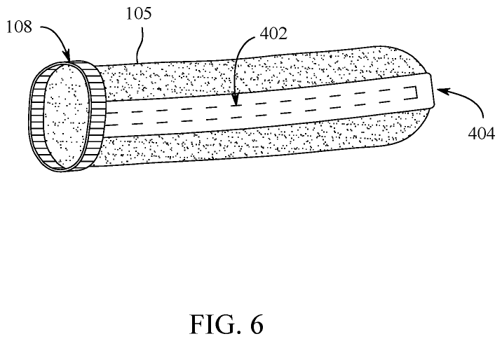

With reference to FIG. 6, a top view of the prophylactic device 400 is shown laid flat on a surface prior to packaging, the device 400 arranged so as to show the flavor ridge 402 running centrally along the entire length of the device 400. The protective strip 404 removably adheres to the flavor ridge 402, and possibly also adheres to portions of the polymeric sleeve 105 that run along the flavor ridge 402.

With reference to FIGS. 1-6, each showing a flavor ridge, a backing layer (not shown) can be applied along the polymeric sleeve wherever a flavor ridge is to be applied. Alternatively, each flavor ridge can be pre-applied to a segment of backing layer, and then the segment of backing with the flavor ridge segment can be applied on the polymeric sleeve wherever a flavor ridge is needed.

The backing layer can include a light adhesive on its back surface that improves the adhesion of the backing layer to the polymeric sleeve. In some embodiments, a front-facing surface of the backing layer also provides a smooth surface that retains its shape while being licked so as to facilitate licking of the flavor ridge off of the backing layer.

The flavor ridges should be placed along and/or over sensitive surfaces of the penis, such as shown in FIGS. 1-6. Other placement and/or arrangement of the flavor ridges can also be effective to provide pleasurable sensations to the penis wearing the prophylactic device while being licked. For example, the flavor ridges can form a series of concentric circles around the circumference of the polymeric sleeve at various points along the polymeric sleeve.

Each flavor ridge of the prophylactic device of the invention is of a thickness that will allow at least ten licks, e.g., 0.25 inches thick. Each flavor ridge should have a generally rounded cross-section on top, and a flat bottom surface due to the adherence of the flavor ridge to the surface of the backing layer, or directly to the surface of the polymeric sleeve.

Each flavor ridge can be made from a hard candy, such as a hard candy similar to Jolly Rancher® hard candy. Thus, a concentrated aqueous solution of the following ingredients can be mixed and then applied to the surface of the backing layer, or directly to the surface of the polymeric sleeve, and allowed to dry: corn syrup, sugar, and no more than 2% of malic acid, natural flavor and/or artificial flavor, artificial color, mineral oil, and lecithin.

Alternatively, a viscous liquid candy, such as a viscous liquid candy similar to Sour Ooze™ Liquid Candy, can be applied to the surface of the backing layer, or directly to the surface of the polymeric sleeve, and allowed to dry. For example, the ingredients of the viscous liquid candy can be: glucose syrup, water, citric acid (sour salt), artificial/natural flavor, sodium benzoate (preservative), and artificial color.

Other modifications and implementations will occur to those skilled in the art without departing from the spirit and the scope of the invention as claimed. Accordingly, the above description is not intended to limit the invention, except as indicated in the following claims.

What is claimed is:

1. A penile prophylactic device for guided fellatio, the device comprising:
a polymeric sleeve having an open end and a closed end, the polymeric sleeve configured to substantially cover a penis of a human male; and
a flavor ridge running along a surface of the polymeric sleeve.

2. The penile prophylactic device for guided fellatio, wherein the flavor ridge runs along a top surface and/or a bottom surface of the polymeric sleeve.

3. The penile prophylactic device of claim 1, wherein the polymeric sleeve is made from a polymeric material selected from one of:
low-density polyethylene (LDPE) or polyurethane or polyisoprene.

4. The penile prophylactic device of claim 1, further including:
an elastic band attached around the circumference of the opening of the polymeric sleeve, the elastic band configured to constrict around a portion of a penis proximal to the scrotum, thereby helping to hold the polymeric sleeve in place along the penis.

5. The penile prophylactic device of claim 1, wherein the elastic band is made of silicone.

6. The penile prophylactic device of claim 1, wherein the flavor ridge includes at least one of:
a sweetening agent, a souring agent, and a flavoring agent.

7. The penile prophylactic device of claim 1, further comprising:
a protective strip removably attached along the flavor ridge, the protective strip being configured to protect freshness of the flavor ridge while the at least one protective strip is attached to the flavor ridge, and configured to be removable so as to leave the flavor ridge substantially in place after the protective strip is removed.

8. The penile prophylactic device of claim 1, wherein the flavor ridge includes a pair of mutually parallel flavor ridges on the polymeric sleeve.

9. The penile prophylactic device of claim 1, further comprising:
a backing layer configured to adhere to the polymeric sleeve, the backing layer applied centrally along the polymeric sleeve,
the flavor ridge being applied centrally along the backing layer.

10. The penile prophylactic device of claim 9, wherein the backing layer includes a coating of light adhesive that improves the adhesion of the backing layer to the polymeric sleeve.

11. The penile prophylactic device of claim 9, wherein the backing layer includes a smooth front-facing surface that is configured to retain its shape while the flavor ridge off is licked off of the backing layer.

12. The penile prophylactic device of claim 1, wherein the device is configured to be packaged in a folded condition.

13. The penile prophylactic device of claim 12, wherein the flavor ridge is folded once or twice when the device is packaged in the folded condition.

14. The penile prophylactic device of claim 1, wherein the flavor ridge is flavored to resemble at least one of:
lemon, lime, orange, cherry, pineapple, grape, blueberry, licorice, chocolate, cotton candy.

15. A penile prophylactic device for enhancing fellatio, the device comprising:
a polymeric sleeve having an open end and a closed end, the polymeric sleeve configured to substantially cover a penis of a human male;
a flavor ridge running along a top surface and/or a bottom surface of the polymeric sleeve; and
an elastic band attached around the circumference of the opening of the polymeric sleeve, the elastic band configured to constrict around a portion of a penis proximal to the scrotum, thereby helping to hold the polymeric sleeve in place along the penis.

16. The penile prophylactic device of claim 15, wherein the polymeric sleeve is made from a polymeric material selected from one of: low-density polyethylene (LDPE) or polyurethane or polyisoprene.

17. The penile prophylactic device of claim 15, wherein the elastic band is made of silicone.

18. The penile prophylactic device of claim 15, wherein the flavor ridge includes at least one of:
a sweetening agent, a souring agent, and a flavoring agent.

19. The penile prophylactic device of claim 15, further comprising:
a protective strip removably attached along the flavor ridge, the protective strip configured to protect freshness of the flavor ridge while the at least one protective strip is attached to the flavor ridge, and configured to be removable so as to leave the flavor ridge substantially in place after the protective strip is removed.
